(19) United States
(12) Patent Application Publication
(10) Pub. No.: US 2018/0335467 A1
(43) Pub. Date: Nov. 22, 2018

(54) TESTING METHOD AND TESTING APPARATUS

(71) Applicant: Mitsubishi Electric Corporation, Tokyo (JP)

(72) Inventors: Fukutaro KATO, Tokyo (JP); Tsuyoshi AMIMOTO, Tokyo (JP); Ryota KURIYAMA, Tokyo (JP); Ryuichi NISHIURA, Tokyo (JP); Satoru TOYAMA, Tokyo (JP); Kota MIZUNO, Tokyo (JP)

(73) Assignee: Mitsubishi Electric Corporation, Tokyo (JP)

(21) Appl. No.: 15/730,056

(22) Filed: Oct. 11, 2017

(30) Foreign Application Priority Data
May 22, 2017 (JP) 2017-100688

(51) Int. Cl. G01R 31/12; G01N 33/28

(52) U.S. Cl. CPC G01R 31/1281; G01N 33/28

(57) ABSTRACT

A testing method of the present invention is a testing method for examining a degree of deterioration, due to oxygen, of an object material constituting an oil-filled electrical apparatus. The oil-filled electrical apparatus is an open-type oil-filled electrical apparatus including an insulating oil, an insulator, and a conductor, the insulating oil being contained as being in contact with the atmosphere. The object material is at least any of the insulating oil, the insulator, and the conductor. The testing method includes putting the insulating oil and the object material into a testing tank, keeping a state where dry air is continuously supplied to the upper space in the testing tank, and subsequently performing measurement of an index of deterioration of the object material due to oxygen.

### BACKGROUND OF THE INVENTION

### Field of the Invention

The present invention relates to a keying method and a testing apparatus.

### Description of the Background Art

It is known that an insulating oil and an insulator and the like, constituting an oil-filled electrical apparatus, such as an oil-filled transformer, deteriorates with age and causes the oil-filled electrical apparatus to reach its life span. It is also known that an insulating oil and an insulator and the like, deteriorates with age due to the influences of, for example, thermal stresses during operation, oxygen supplied from outside into the insulating oil and moisture in the insulator (for example, NPD 1: M L. Couilibaly, C. Perrier, M. Marugan, “Assessment of Methanol as cellulose aging marker in mineral and ester oils”, CIGRE Conference Paris, A2-112, 2016).

In particular, oxygen is a factor that has a great influence on the deterioration with age of an insulating oil and an insulator and the like. Accordingly, in order to accurately evaluate life span of an insulating oil and an insulator and the like, it is desired to examine the influence of oxygen with a testing system equivalent to an actual apparatus (actual oil-filled electrical apparatus).

As an oil-filled electrical apparatus, an sealed type with a low oxygen permeation into an insulating oil and an open type with a high oxygen permeation into an insulating oil are known (for example, PTD 1: Japanese Patent Laying-Open No. 2010-27634). Examples of a known testing system to simulate oxygen permeation into an insulating oil in an open-type oil-filled electrical apparatus include a method of performing a test by dissolving air in an insulating oil first and then sealing the container, and a method with continuous air bubbling in an insulating oil.

### SUMMARY OF THE INVENTION

With the conventional testing methods, however, the influence of oxygen in an actual apparatus cannot be satisfactorily simulated, and thus an accurate evaluation cannot be made for the influence of oxygen on the deterioration with age of an insulating oil and an insulator and the like, in an oil-filled electrical apparatus.

Therefore, an object of the present invention is to provide a testing method that can make an accurate evaluation for the influence of oxygen on the deterioration with age of an insulating oil and an insulator and the like, in an oil-filled electrical apparatus.

A testing method of the present invention is a testing method for examining a degree of deterioration, due to oxygen, of an object material constituting an oil-filled electrical apparatus.

The oil-filled electrical apparatus is an open-type oil-filled electrical apparatus including an insulating oil, an insulator, and conductor, the insulating oil being contained as being in contact with the atmosphere.

The object material is at least any of the insulating oil, the insulator, and the conductor.

The testing method includes putting the insulating oil and the object material into a testing tank, keeping a state where dry air is continuously supplied to the upper space in the testing tank, and subsequently performing measurement of an index of deterioration of the object material due to oxygen.

According to the present invention, a testing method can be provided that can make an accurate evaluation for the Influence of oxygen on the deterioration with age of an insulating oil and an insulator and the like, in an oil-filled electrical apparatus.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic diagram showing a testing apparatus according to Embodiment 1.

FIG. 2 is a schematic diagram showing a testing apparatus of Reference Examples 1 and 2.

FIG. 3 is a schematic diagram showing another testing apparatus usable for reference examples.

FIG. 4A is a graph showing measurement of Example 1.

FIG. 4B is a graph showing measurements of Reference Example 1.

FIG. 5A is a graph showing measurements of Example 2.

FIG. 5B is a graph showing measurements of Reference Example 2.

### DESCRIPTION OF THE PREFERRED EMBODIMENTS

Embodiments of the present invention are described below with reference to the drawings. The drawings, identical reference numerals denote identical or equivalent parts.

### Embodiment 1

The testing method of the present embodiment is a testing method for examining a degree of deterioration of an object material constituting an oil-filled electrical apparatus. The oil-filled electrical apparatus is an open-type oil-filled electrical apparatus including an insulating oil, an insulator, and a conductor, the insulating oil being contained as being in contact with the atmosphere.

The oil-filled electrical apparatus may be any electrical apparatus that includes an insulating oil, an insulator, and a conductor. In the oil-filled electrical apparatus, the insulator and the conductor are preferably immersed in the insulating oil. The oil-filled electrical apparatus is preferably a transformer.

The object material is at least any of the insulating oil, the insulator, and the conductor used for the oil-filled electrical apparatus.

The insulating oil may he any insulating oil that is usable for the oil-filled electrical apparatus. Examples of the insulating oil include mineral oils, silicone oils, and ester oils.

The insulator may be any insulator (insulating body) that is usable for the oil-filled electrical apparatus. Examples of the insulator include insulating paper, such as pressboard (PB), kraft paper, and thermally upgraded paper.

The conductor may be any conductor that is usable for the oil-filled electrical apparatus. Examples of the conductor include copper. The conductor is used for, for example, a coil of the transformer (oil-filled electrical apparatus).

In the testing method of the present embodiment, the insulating oil and the object material are put into a testing tank, a state where dry air is continuously supplied to the upper space in the testing tank is kept, and then measurement is performed of an index of deterioration of the object material due to oxygen.

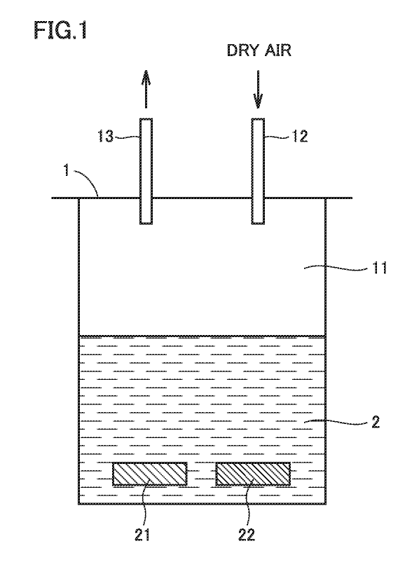

With reference to FIG. 1, putting the insulating oil and the object material into a testing tank 1 includes putting only an insulating oil 2 in testing tank 1 if the object material includes only the insulating oil. If the object material includes a material other than the insulating oil insulating oil 2 and an object material other than the insulating oil (at least any of an insulator 21 and a conductor 22) are put into the testing tank. Thus, in testing tank 1, insulating oil 2 is always put and a material other than the insulating oil is put depending on the object material (in FIG. 1, insulator 21 and conductor 22 do not necessarily have to be put).

As a method of continuously supplying dry air to the upper space in the testing tank, there is, for example, a method where dry air is supplied from an inlet 12 to an upper space 11 with, for example, a pump, and where dry air is discharged from an outlet 13.

The reason for using dry air is that, in an actual open-type apparatus, such as a transformer, a moisture remover (for example, silica gel) is provided at an air inlet to dry the air in contact with the insulating oil.

A retention time, which is the duration of a state where dry air is continuously supplied to the upper space in the testing tank, is not particularly limited but may be set as appropriate in accordance with, for example, the measuring limit of an index of deterioration. In the present embodiment, the insulating oil may be heated while dry air is continuously supplied to the upper space in the testing tank. In such a case, the deterioration of the object material is accelerated and the retention time can be shortened. Thus, the test can be completed in a short time.

If the object material is an insulating oil, examples of the index of deterioration of the insulating oil due to oxygen include the level of electrification of the insulating oil, and the concentration of a dissolved gas (carbon monoxide, carbon dioxide) in the insulating oil. If the object material is insulating paper (insulator), examples of the index of deterioration of the object material due to oxygen include the average degree of polymerization of the insulating paper (cellulose molecules constituting the insulating paper), and the content of furfural in the insulating oil, the furfural being a decomposition product of the insulating paper generated due to the presence of oxygen. If the object material is insulating paper (insulator) and a conductor, examples of the index of deterioration of the object material due to oxygen include the corrosion (the amount of, for example, copper sulfide produced) of the insulating paper and the conductor immersed in the insulating oil, with the conductor being wound around the insulating paper.

The present embodiment also relates to a testing apparatus usable for the testing method described above. With reference to FIG. 1, the testing apparatus includes testing tank 1 for containing an insulating oil. Testing tank 1 includes, for example, inlet 12 and outlet 13 to continuously supply dry air to upper space 11 (the space above the oil surface of insulating oil 2 in testing tank 1) in testing tank 1.

With a conventional method of performing a test by dissolving air in an insulating oil first and then sealing the container, the apparatus is different from an actual apparatus in condition alter the oxygen in the first dissolved air has been consumed. Thus, an accurate evaluation cannot be made for the influence of oxygen on the deterioration with age of an insulating oil and an insulator and the like. Also, with a method with continuous air bubbling in an insulating oil, the amount of oxygen dissolved into the insulating oil is large than that of an actual apparatus. Thus, the influence of oxygen on the deterioration with age of an insulating oil and an insulator and the like, is overestimated and cannot be accurately evaluated.

According to the present embodiment, in contrast, the test is performed in a state closer to that of an actual apparatus compared to the conventional methods. Therefore, an accurate evaluation can be made for the influence of oxygen on the deterioration with age of an insulating oil and an insulator and the like, in an open-type oil-filled electrical apparatus.

### EXAMPLE

### Example 1

The present example relates to an example test using a testing system where the insulating oil is exposed to air as with an actual apparatus. A deterioration test for an insulator was performed using the testing apparatus described above with reference to FIG. 1 for Embodiment 1.

An insulating oil was put into a testing tank, and pressboard (PB), kraft paper, or thermally upgraded paper was immersed in the insulating oil as an insulator. Unlike FIG. 1, conductor 22 was not immersed in insulating oil 2 in the present example.

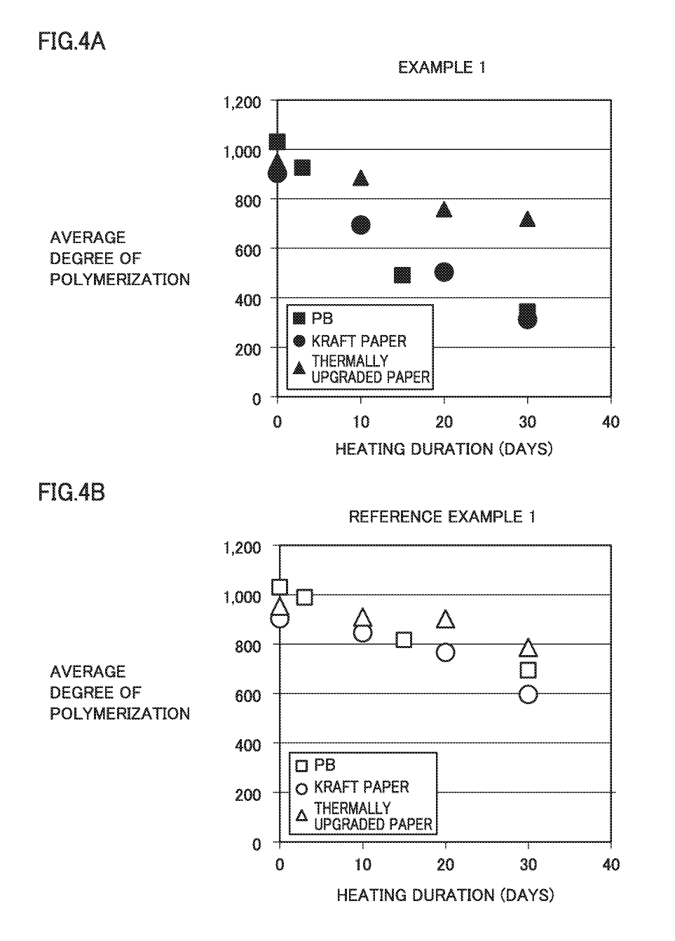

An accelerated deterioration test for the insulator by heat was performed while dry air (gas mixture of 80 volume % nitrogen and 20 volume % oxygen) or nitrogen was continuously flowing through the upper space in the testing tank (the space above the oil surface of the insulating oil). The average degree of polymerization of the insulator was then measured (monitored). As for PB, measurement was performed at the points of time at which the retention time was 0, 3, 15, and 30 days. As for kraft paper and thermally upgraded paper, measurement was performed at the points of time at which the retention time was 0, 10, 20, and 30 days. The change over time in average degree of polymerization is shown in FIG. 4A.

### Reference Example 1

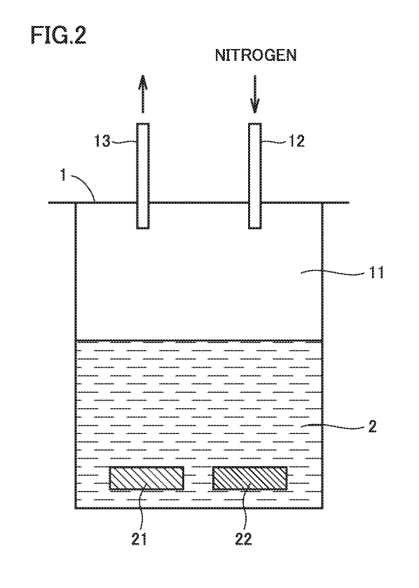

The present reference example relates to an example test using a test system where the insulating oil is not exposed to oxygen. In the present reference example, the average degree of polymerization of an insulator was measured in a similar manner to Example 1 except that nitrogen was used instead of dry air in the present reference example, as shown in FIG. 2. The change over time in average degree of polymerization is shown in FIG. 4B.

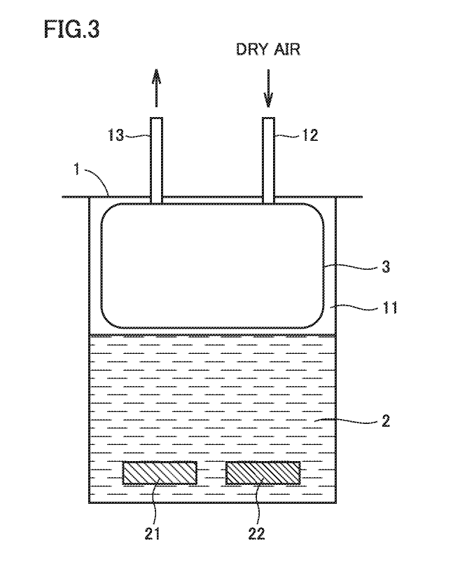

As a testing system where the insulating oil is not exposed to oxygen, a testing system (testing apparatus) having a rubber bag 3 contained in upper space 11 so that dry air is supplied to the inside of rubber bag 3 may be used, as shown in FIG. 3, for example.

### Example 2

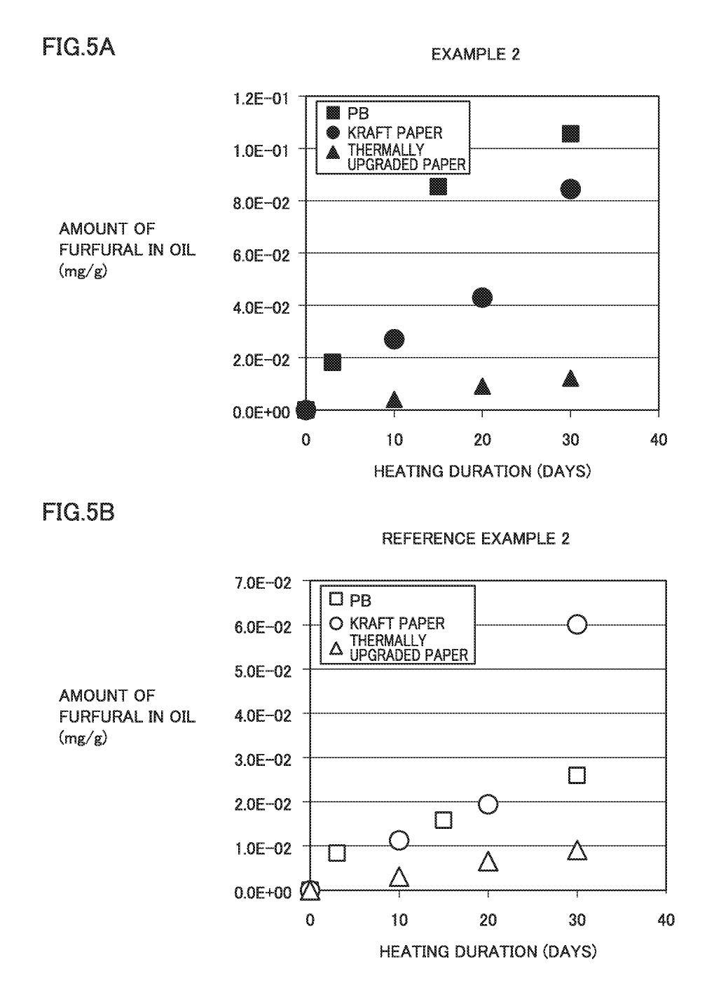

Instead of the average degree of polymerization of the insulator, the amount (concentration) of furfural in the insulating oil was measured (monitored). Otherwise, Example 2 is similar to Example 1. The change over time in amount of furfural is shown in FIG. 5A.

### Reference Example 2

In the present reference example, the amount of furfural was measured in a similar manner to Example 2 except that nitrogen was used instead of dry air in the present reference example, as shown in FIG. 2. The change over time in amount of furfural is shown in FIG. 5B.

From the results shown in FIG. 4A, FIG. 4B, FIG. 5A, and FIG. 5B, when a comparison is made with respect to the point of time when the retention time (heating duration) is 30 days, it is found that, as for PB and kraft paper, the rate of decline in average degree of polymerization is about 2.0 times due to the presence of oxygen. Specifically, the average degree of polymerization declines by 639 (the average value of PB and kraft paper) from the 0th day of heating with oxygen, and declines by 321 without oxygen. That is, the rate of decline in average degree of polymerization is 639/321=2.0 times due to the presence of oxygen. Further, it is also found that the amount of furfural is about 2.1 times. Specifically, the amount of furfural is 0.094 mg/g (the average value PB and kraft paper) with oxygen, and is 0.045 mg/g without oxygen. That is, the rate of generation of furfural is 0.094/0.045=2.1 times due to the presence of oxygen. As for thermally upgraded paper, on the other hand, it is found that the rate of decline in average degree of polymerization is about 1.4 due to the presence of oxygen. Specifically, the average degree of polymerization declines by 234 from the 0th day of heating with oxygen, and declines by 167 without oxygen. That is, the rate of decline in average degree of polymerization is 234/167=1.4 times due to the presence of oxygen. Further, it is also found that the amount of furfural is about 1.3 times. Specifically, the amount of furfural is 0.012 mg/g with oxygen, and is 0.009 mg/g without oxygen. That is, the rate of generation of furfural is 0.012/0.009=1.3 times due to the presence of oxygen.

From these results, it is found that PB and kraft paper deteriorate more easily due to the presence of oxygen than thermally upgraded paper. In this way, the influence of oxygen on the deterioration properties of an insulator can be evaluated.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A testing method for examining a degree of deterioration, due to oxygen, of an object material constituting an oil-filled electrical apparatus,
the oil-filled electrical apparatus being an open-type oil-filled electrical apparatus including an insulating oil, an insulator, and a conductor, the insulating oil being contained as being in contact with atmosphere,
the object material being at least any of the insulating oil, the insulator, and the conductor,
the testing method comprising:
putting the insulating oil and the object material into a testing tank;
keeping a state where dry air is continuously supplied to an upper space in the testing tank; and
subsequently performing measurement of an index of deterioration of the object material due to oxygen.

2. The testing method according to claim 1, wherein the insulating oil is heated while the dry air is continuously supplied to the upper space in the testing tank.

3. The testing method according to claim 1, wherein the oil-filled electrical apparatus is a transformer.

4. A testing apparatus usable for the testing method according to claim 1, comprising the testing tank for containing the insulating oil,
the testing tank including an inlet and an outlet to continuously supply the dry air to the upper space in the testing tank.
